(19) United States
(12) Patent Application Publication
(10) Pub. No.: US 2021/0196322 A1
(43) Pub. Date: Jul. 1, 2021

(54) Foot Sander Assembly

(71) Applicant: Candace Fletcher, Chicago, IL (US)

(72) Inventor: Candace Fletcher, Chicago, IL (US)

(21) Appl. No.: 16/728,463

(22) Filed: Dec. 27, 2019

(51) Int. Cl. A61B 17/54; A61B 17/32; A45D 29/00

(52) U.S. Cl. CPC A61B 17/54; A61B 17/32002; A61B 2017/00424; A61B 2017/320004; A45D 29/00

(57) ABSTRACT

A foot sander assembly includes a sanding plate that is positionable against the bottom of a user's foot. A handle is coupled to the sanding plate for gripping. An oscillator is positioned in the sanding plate and the oscillator oscillates when the oscillator is turned on. Moreover, the oscillator is in mechanical communication with the sanding plate thereby facilitating the sanding plate to vibrate when the oscillator is turned on. A sanding pad is attachable to the sanding plate. The sanding pad is comprised of an abrasive material to abrade dead skin from the bottom of the user's foot when the oscillator is turned on.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to sander devices and more particularly pertains to a new sander device for removing dead skin from the bottom of a user's foot.

### (2) Description of Related Art Including Information Disclosed Under 37 CFR 1.97 and 1.98

The prior art relates to sander devices.

### BRIEF SUMMARY OF THE INVENTION

An embodiment of the disclosure meets the needs presented above by generally comprising a sanding plate that is positionable against the bottom of a user's foot. A handle is coupled to the sanding plate for gripping. An oscillator is positioned in the sanding plate and the oscillator oscillates when the oscillator is turned on. Moreover, the oscillator is in mechanical communication with the sanding plate thereby facilitating the sanding plate to vibrate when the oscillator is turned on. A sanding pad is attachable to the sanding plate. The sanding pad is comprised of an abrasive material to abrade dead skin from the bottom of the user's foot when the oscillator is turned on.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a top perspective view of a foot sander assembly according to an embodiment of the disclosure.

FIG. 2 is a left side phantom view of an embodiment of the disclosure.

FIG. 3 is a bottom phantom view of an embodiment of the disclosure.

FIG. 4 is a bottom perspective view of an embodiment of the disclosure showing a sanding pad being applied to a sanding plate.

### DETAILED DESCRIPTION OF THE INVENTION

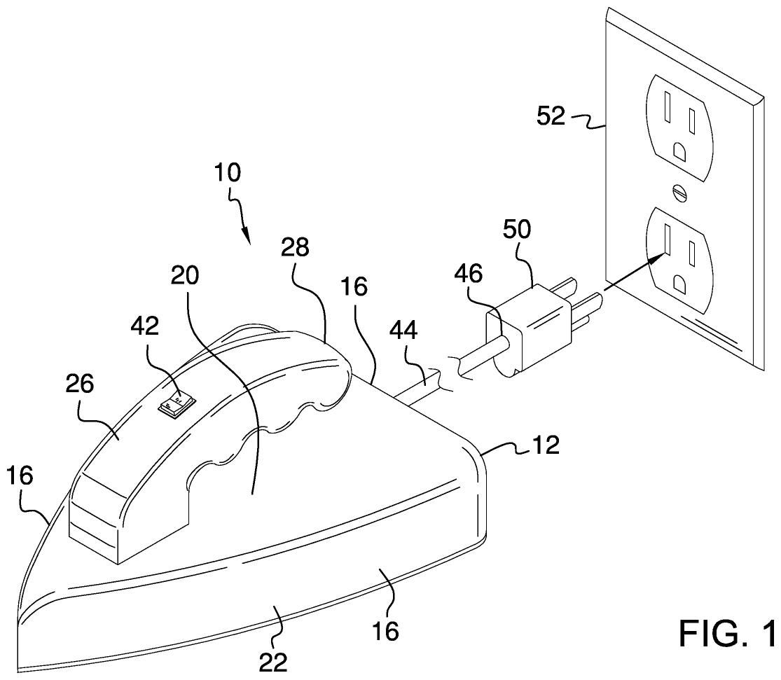

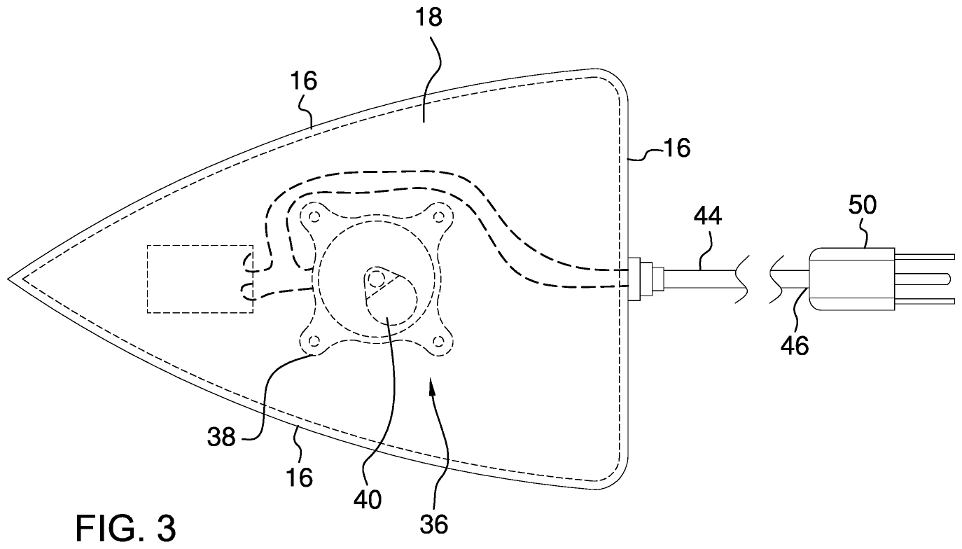

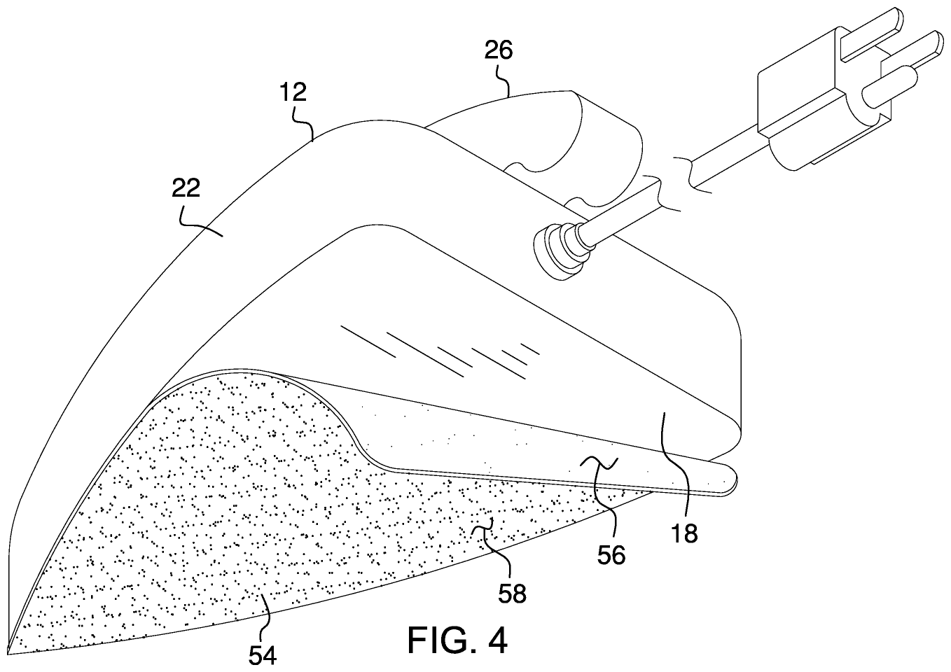

With reference now to the drawings, and in particular to FIGS. 1 through 4 thereof, a new sander device embodying the principles and concepts of an embodiment of the disclosure and generally designated by the reference numeral 10 will be described.

As best illustrated in FIGS. 1 through 4, the foot sander assembly 10 generally comprises a sanding plate 12 that is positionable against the bottom of a user's foot. A perimeter of the sanding plate 12 has a plurality of intersecting sides 16 such that the sanding plate 12 has a triangular shape. The sanding plate 12 has a bottom wall 18, a top wall 20 and a perimeter wall 22 extending therebetween, and each of the intersecting sides 16 of the perimeter of the sanding plate 12 is associated with the perimeter wall 22. Moreover, the sanding plate 12 has a chamber 24 extending between the top wall 20 and the bottom wall 18.

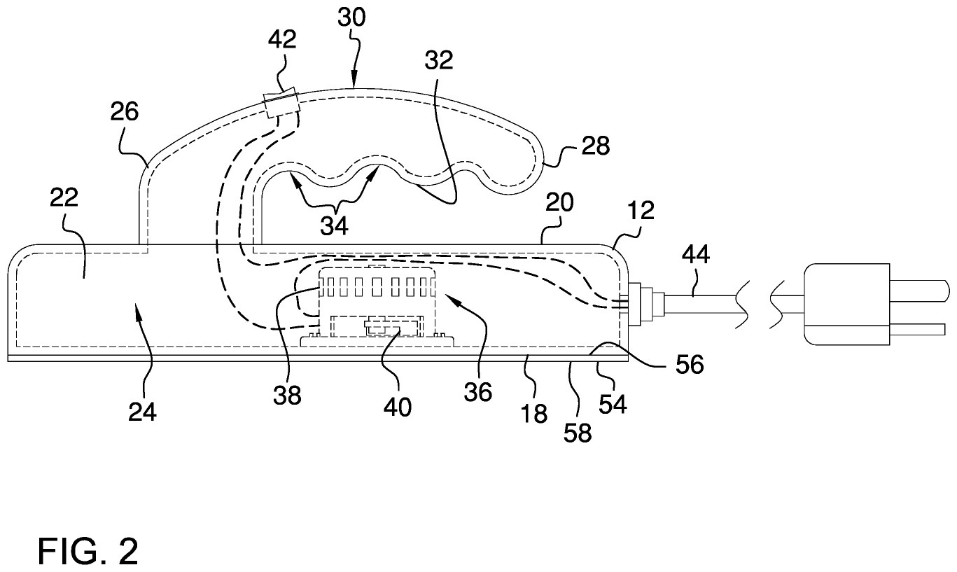

A handle 26 is coupled to the sanding plate 12 for gripping. The handle 26 is positioned on the top wall 20 of the sanding plate 12 and the handle 26 has a distal end 28 with respect to the top wall 20. Additionally, the handle 26 curves between the top wall 20 and the distal end 28 to define a grip portion 30 of the handle 26 that is spaced from and extends along the top wall 20. The grip portion 30 of the handle 26 has a lower surface 32 facing the top wall 20 of the sanding plate 12. The lower surface 32 has a plurality of indentations 34 thereon. The indentations 34 are spaced apart from each other and are distributed along the grip portion 30. Each of the indentations 34 accommodates a respective one of the user's fingers when the handle 26 is gripped. As is most clearly shown in FIG. 2, the handle 26 may be hollow and the handle 26 may be in fluid communication with the chamber 24 in the sanding plate 12.

An oscillator 36 is provided and the oscillator 36 is positioned in the sanding plate 12. The oscillator 36 oscillates when the oscillator 36 is turned on. Moreover, the oscillator 36 is in mechanical communication with the sanding plate 12 thereby facilitating the sanding plate 12 to vibrate when the oscillator 36 is turned on. The oscillator 36 comprises a motor 38 that is positioned within the chamber 24 in the sanding plate 12. The motor 38 rotates in a first direction when the motor 38 is turned on. Additionally, the motor 38 is coupled to the bottom wall 18 of the sanding plate 12. The motor 38 may be an electric motor or the like.

The oscillator 36 includes a cam 40 that is rotatably coupled to the motor 38. The cam 40 is offset from a rotational axis of the motor 38 such that the cam 40 oscillates when the motor 38 rotates in the first direction. In this way the vibration produced by the oscillation is transferred into the bottom wall 18 of the sanding plate 12. A switch 42 is movably coupled to the handle 26 and the switch 42 is electrically coupled to the motor 38. The switch 42 is positionable in an on position or an off position. The motor 38 is turned on when the switch 42 is in the on position and the motor 38 is turned off when the switch 42 is in the off position.

A power cord 44 is coupled to and extends away from the perimeter wall 22 of the sanding plate 12 and the power cord 44 is electrically coupled to the motor 38. The power cord 44 has a distal end 46 with respect to the perimeter wall 22 and a plug 50 is electrically coupled to the distal end 46. The plug 50 is pluggable into a power source 52 comprising a female electrical outlet. The plug 50 may be a three prong male electrical plug or other similar electrical plug.

A sanding pad 54 is provided and the sanding pad 54 is attachable to the sanding plate 12. Thus, the sanding pad 54 can be positioned to abut the bottom of the user's foot. The sanding pad 54 is comprised of an abrasive material. In this way the sanding pad 54 can abrade dead skin from the bottom of the user's foot when the oscillator 36 is turned on. The sanding pad 54 has a top surface 56 and a bottom surface 58, and the top surface 56 is comprised of an adhesive material. In this way the top surface 56 adheres to the bottom wall 18 of the sanding plate 12 having the bottom surface 58 being exposed. The bottom surface 58 is comprised of an abrasive material that would commonly be used with sandpaper or other material employed to safely abrade human skin.

In use, the sanding pad 54 is adhered to the bottom wall 18 of the sanding plate 12. The switch 42 is positioned in the on position thereby facilitating the oscillator 36 to vibrate the sanding plate 12. The sanding pad 54 is placed against the bottom of the user's foot, or other preferred location on the user's body. In this way the sanding pad 54 abrades the bottom of the user's foot when the oscillator 36 is turned on. Thus, the user can enhance the health and appearance of the bottom of the user's feet.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A foot sander assembly being configured to abrade dead skin from the bottom of a user's foot, said assembly comprising:
a sanding plate being positionable against the bottom of a user's foot, a perimeter of said sanding plate having a plurality of intersecting sides such that said sanding plate has a triangular shape;
a handle being coupled to said sanding plate for gripping;
an oscillator being positioned in said sanding plate, said oscillator oscillating when said oscillator is turned on, said oscillator being in mechanical communication with said sanding plate thereby facilitating said sanding plate to vibrate when said oscillator is turned on; and
a sanding pad being attachable to said sanding plate wherein said sanding pad is configured to abut the bottom of the user's foot, said sanding pad being comprised of an abrasive material wherein said sanding pad is configured to abrade dead skin from the bottom of the user's foot when said oscillator is turned on.

2. The assembly according to claim 1, wherein said sanding plate has a bottom wall, a top wall and a perimeter wall extending therebetween, each of said intersecting sides of said perimeter of said sanding plate being associated with said perimeter wall, said sanding plate having a chamber extending between said top wall and said bottom wall.

3. The assembly according to claim 2, wherein said handle is positioned on said top wall of said sanding plate, said handle having a distal end with respect to said top wall, said handle curving between said top wall and said distal end to define a grip portion of said handle being spaced from and extending along said top wall, said grip portion of said handle having a lower surface facing said top wall of said sanding plate, said lower surface having a plurality of indentations thereon, said indentations being spaced apart from each other and being distributed along said grip portion wherein each of said indentations is configured to accommodate a respective one of the user's fingers when said handle is gripped.

4. The assembly according to claim 2, wherein said oscillator comprises a motor being positioned within said chamber in said sanding plate, said motor rotating in a first direction when said motor is turned on, said motor being coupled to said bottom wall of said sanding plate.

5. The assembly according to claim 4, further comprising a cam being rotatably coupled to said motor, said cam being offset from a rotational axis of said motor such that said cam oscillates when said motor rotates in said first direction thereby transferring vibration into said bottom wall of said sanding plate.

6. The assembly according to claim 5, further comprising a switch being movably coupled to said handle, said switch being electrically coupled to said motor, said switch being positionable in an on position or an off position, said motor being turned on when said switch is in said on position, said motor being turned off when said switch is in said off position.

7. The assembly according to claim 6, further comprising a power cord being coupled to and extending away from said perimeter wall of said sanding plate, said power cord being electrically coupled to said motor, said power cord having a distal end with respect to said perimeter wall, said distal end having a plug being electrically coupled thereto, said plug being pluggable into a power source comprising a female electrical outlet.

8. The assembly according to claim 3, wherein said sanding pad has a top surface and a bottom surface, said top surface being comprised of an adhesive material, said top surface adhering to said bottom wall of said sanding plate having said bottom surface being exposed, said bottom surface being comprised of said abrasive material.

9. A foot sander assembly being configured to abrade dead skin from the bottom of a user's foot, said assembly comprising:
a sanding plate being positionable against the bottom of a user's foot, a perimeter of said sanding plate having a plurality of intersecting sides such that said sanding plate has a triangular shape, said sanding plate having a bottom wall, a top wall and a perimeter wall extending therebetween, each of said intersecting sides of said perimeter of said sanding plate being associated with said perimeter wall, said sanding plate having a chamber extending between said top wall and said bottom wall;
a handle being coupled to said sanding plate for gripping, said handle being positioned on said top wall of said sanding plate, said handle having a distal end with respect to said top wall, said handle curving between said top wall and said distal end to define a grip portion of said handle extending along a line being oriented parallel to said top wall, said grip portion of said handle having a lower surface facing said top wall of said sanding plate, said lower surface having a plurality of indentations thereon, said indentations being spaced apart from each other and being distributed along said grip portion wherein each of said indentations is configured to accommodate a respective one of the user's fingers when said handle is gripped;
an oscillator being positioned in said sanding plate, said oscillator oscillating when said oscillator is turned on, said oscillator being in mechanical communication with said sanding plate thereby facilitating said sanding plate to vibrate when said oscillator is turned on, said oscillator comprising:
a motor being positioned within said chamber in said sanding plate, said motor rotating in a first direction when said motor is turned on, said motor being coupled to said bottom wall of said sanding plate;
a cam being rotatably coupled to said motor, said cam being offset from a rotational axis of said motor such that said cam oscillates when said motor rotates in said first direction thereby transferring vibration into said bottom wall of said sanding plate;
a switch being movably coupled to said handle, said switch being electrically coupled to said motor, said switch being positionable in an on position or an off position, said motor being turned on when said switch is in said on position, said motor being turned off when said switch is in said off position; and
a power cord being coupled to and extending away from said perimeter wall of said sanding plate, said power cord being electrically coupled to said motor, said power cord having a distal end with respect to said perimeter wall, said distal end having a plug being electrically coupled thereto, said plug being pluggable into a power source comprising a female electrical outlet; and
a sanding pad being attachable to said sanding plate wherein said sanding pad is configured to abut the bottom of the user's foot, said sanding pad being comprised of an abrasive material wherein said sanding pad is configured to abrade dead skin from the bottom of the user's foot when said oscillator is turned on, said sanding pad having a top surface and a bottom surface, said top surface being comprised of an adhesive material, said top surface adhering to said bottom wall of said sanding plate having said bottom surface being exposed, said bottom surface being comprised of said abrasive material.
